(19) United States
(12) Patent Application Publication
(10) Pub. No.: US 2021/0239638 A1
(43) Pub. Date: Aug. 5, 2021

(54) METHOD FOR FABRICATION OF MEMS INTEGRATED SENSOR AND SENSOR THEREOF

(71) Applicant: Indian Institute of Technology, Delhi, New Delhi (IN)

(72) Inventors: Saakshi Dhanekar, New Delhi (IN); Priyanka Dwivedi, New Delhi (IN)

(21) Appl. No.: 17/048,662

(22) PCT Filed: Apr. 17, 2019
(86) PCT No.: PCT/IB2019/053185

§ 371 Date: Oct. 19, 2020

(30) Foreign Application Priority Data
Apr. 17, 2018 (IN) 201811014593

(51) Int. Cl. G01N 27/12

(52) U.S. Cl. CPC G01N 27/128; G01N 27/125

(57) ABSTRACT

The present invention provides a method of fabrication and characterization of a wafer scalable, planar MEMS heater integration with TiO₂/Nano-Si as an ethanol sensing platform. Nano-Si with TiO₂ and MEMS heater is introduced, which contains the porous morphology with significant depth. Due to the introduction of Nano-Si in micro heater fabrication the power consumption of the complete chip is significantly reduced. This significant change in power consumption is due to the thermal conductivity of Nano-Si.

### TECHNICAL FIELD OF THE INVENTION

The present subject matter described herein, in general, relates to fabrication of Microelectromechanical systems (MEMS) integrated sensor platform, and more particularly relates to a scalable planar MEMS technology process for low power operated selective ethanol sensor.

### BACKGROUND OF THE INVENTION

Of late, solid state gas sensors are getting popular due to their wide applications, that includes but not limited to, chemical detections, industries, environmental monitoring, healthcare, air quality monitoring systems, and the like.

Reference is made to a non-patent literature, P. Kumar, et.al. The rise of low-cost sensing for managing air pollution in cities, Environ. Int. 75 (n. d.) 199-205. doi:10.1016/j.envint.2014.11.019.

Further, reference is made to a non-patent literature, M. Penza, E. Consortium, COST Action TD1105: Overview of sensor-systems for air-quality monitoring, Procedia Eng. 87 (2014) 1370-1377. doi:10.1016/j.proeng.2014.11.698.

Still further, reference is made to a non-patent literature, W.Y. Yi, et.al. A Survey of Wireless Sensor Network Based Air Pollution Monitoring Systems, 2015. doi:10.3390/s151229859.

Further, reference is made to a non-patent literature, E. G. Snyder, et.al. The Changing Paradigm of Air Pollution Monitoring, (2013). doi:10.1021/es4022602.

Apart from the above application, due to their simple operation, easy electronic interface, good sensitivity, low cost, fast response, capability to detect number of hazardous gases with very low maintenance, they are given vital importance in modern industry and health care.

Reference is made to non-patent literature, C. Wang, et.al. Metal oxide gas sensors: Sensitivity and influencing factors, Sensors. 10 (2010) 2088-2106. doi:10.3390/s100302088.

Reference is made to yet another non-patent literature, S. Capone, et.al. Solid state gas sensors: state of the art and future activities, 5 (2003) 1335-1348.

Further, reference is made to another non-patent literature, X. Liu, et.al. I. Engineering, A Survey on Gas Sensing Technology, (2012) 9635-9665. doi:10.3390/s120709635.

Metal oxides possess a broad range of electronic, chemical, and physical properties that are often highly sensitive to changes in their chemical environment. Sensors that based out of metal oxides are electrical conductivity sensors. The resistance of their active sensing layer (receptor) changes due to contact with the gas to be sensed. Due to their chemical composition, metal oxide gas sensors are well-suited for a wide range of applications and for the detection of all reactive gases.

Reference is made to a non-patent literature, C. Lu, et.al. Multi-field simulations and characterization of CMOS-MEMS high-temperature smart gas sensors based on SOI technology, (2008). doi:10.1088/0960-1317/18/7/075010.

Further, reference is made to a non-patent literature, G. Sberveglieri, Recent developments in semiconducting thin-film gas sensors, 23 (1995) 103-109.

Based on the electronic structure, metal oxide semiconductor elements, that includes but not limited to, ZnO, SnO₂, In₂O₃, WO₃, TiO₂, V₂O₃, MoO₃, etc. are suitable for detecting, reducing or oxidising gases through conductive measurements. But all these metal oxides based sensors work at high temperatures and always suffer from the selectivity issues. Due to their high operating temperatures, metal oxides based sensors are not suitable for battery operated systems.

Reference is made to a non-patent literature, L. Zhu, W. Zeng, Sensors and Actuators A: Physical Room-temperature gas sensing of ZnO-based gas sensor: A review, 267 (2017) 242-261.

Reference is further made to a non-patent literature, M. Kumar, et.al., SnO₂ based sensors with improved sensitivity and response—recovery time, 40 (2014) 8411-8418. doi:10.1016/j.ceramint.2014.01.050.

Still further, reference is made to a non-patent literature, Z. Zhan, et.al., Highly selective ethanol In₂O₃-based gas sensor, 42 (2007) 228-235. doi:10.1016/jmaterresbull.2006.06.006.

Further, reference is made to a non-patent literature, M. Hubner, et.al., Sensors and Actuators B: Chemical CO sensing mechanism with WO₃ based gas sensors, 151 (2010) 103-106. doi: 10.1016/j.snb.2010.09.040.

Still further, reference is made to a non-patent literature, J. Nisar, et. al., TiO₂-Based Gas Sensor: A Possible Application to SO₂, 2 (2013). doi:10.1021/am4018835.

Further, reference is made to a non-patent literature, Y. Liang, Y. Cheng, Combinational physical synthesis methodology and crystal features correlated with oxidizing gas detection ability of one-dimensional ZnO-VOₓ crystalline hybrids CrystEngComm. 17 (2015) 5801-5807. doi:10.1039/C5CE01110H.

Further, reference is made to a non-patent literature, P. Dwivedi, S. Dhanekar, S. Das, Synthesis of α-MoO₃ nano-flakes by dry oxidation of RF sputtered Mo thin films and their application in gas sensing, 31 (2016) 115010.

However, nowadays, the researchers are working towards smart sensors, for which there is a need for a low temperature, low power consumption, IC compatible micro-fabrication process. Therefore, MEMS technology came for these type of device solutions. But thermal insulation is an important concern in MEMS technology, which is directly related to the power consumption of the MEMS based sensor.

In order to overcome this problem, there is a need to provide low dimension devices or/and make changes in the sensor platform in order to reduce the power consumption. For prior art disclosing, MEMS based sensor operating at high temperature with high power consumption, reference is made to a non-patent literature, B. Behera, S. Chandra, A MEMS based acetone sensor incorporating ZnO nanowires synthesized by wet oxidation of Zn film, J. Micromechanics Microengineering. 25 (2015) 15007. doi:10.1088/0960-1317/25/1/015007. Further, reference is also made to, W. Hwang, K. Shin, J. Roh, D. Lee, S. Choa, C. Sensor, B. Seongnam, Development of Micro-Heaters with Optimized Temperature Compensation Design for Gas Sensors, (2011) 2580-2591. doi:10.3390/s110302580. These prior art MEMS based sensors have been designed for various applications with different requirements, each with its own possibilities and limitations.

Since, Nano-Si is an interesting example of nanostructured material in sensors due to simple wafer scale fabrication, easy to integrate for system on chip (SOC), low cost, high surface area, porosity, sensitivity, IC compatibility, etc. Therefore, it opens a new door for unique and new opportunities for different research areas with applications, that include but not limited to, sensors, biotechnology, medicine, optoelectronics, and chemistry and so forth.

Reference is made to a non-patent literature document, G. F. Fine, L. M. Cavanagh, A. Afonja, R. Binions, Metal Oxide Semi-Conductor Gas Sensors in Environmental Monitoring, (2010) 5469-5502. doi:10.3390/s100605469.

Further, reference is made to a non-patent literature document, M. Righettoni, A. Amann, S. E. Pratsinis, Breath analysis by nanostructured metal oxides as chemo-resistive gas sensors, Biochem. Pharmacol. 18 (2015) 163-171. doi:10.1016/j.mattod.2014.08.017.

Still further, reference is made to a non-patent literature document, C. B. Wilson, Sensors in medicine, 319 (1999) 13-15.

Further, the nanostructured material in sensors has low thermal conductivity which can be used a key feature in development of different micro heater designs operating at low voltages and consuming less power. Although nano -Si does not provide an operative insulation to micro heater device (used thermal oxide under layer), it has additional advantages. With these major features there is a tradeoff between thermal conductivity and IC compatibility feature, can definitely be worthwhile for specific applications. The key issue is how excellently the integration of micro heater platforms and nanostructured materials, with low power consumption can be done.

Reference is made to a patent document, U.S. Pat. No. 6,161,421A that discloses the integration of a SiC heater of comb or finger electrode shape and a SnO₂ thin film gas sensing element applied over distinct portions on the same Si substrate together with Al₂O₃ and SnO₂ thin films via a VLSI technology. It further discloses operating temperature of the sensor to be around 300-400° C. and a heater is made underneath the sensing film. But such high temperature parameters are difficult to maintain.

Reference is made to another patent document, U.S. Pat. No. 6,862,919B2 that discloses a method for forming the sensor comprising disposing capacitance electrodes and a heater on green layers; disposing the layers such that the capacitance electrodes are disposed between adjacent green layers and the heater is disposed on a side of a green layer opposite one of the capacitance electrodes; disposing a gap insert in physical contact with the capacitance electrodes, wherein the gap insert has a higher sintering temperature than the green layers; sintering the green layers; and removing the gap insert.

Reference is made to a non-patent literature, ‘MoO₃/Nano—Si Heterostructure based Highly Sensitive and Acetone Selective Sensor Prototype: A key to Non-invasive Detection of Diabetes, P. Dwivedi et al., IOP Science, 10 Apr. 2018’, that presents development of extremely sensitive and selective acetone sensor prototype which can be used as platform for non-invasive diabetes detection through exhaled human breath. The miniaturized sensors were produced in high yield with the use of standard micro-fabrication processes. Sensors were based on a heterostructure comprising of MoO₃ and nano-porous silicon (NPS). An acetone selective, enhanced sensor response and 0.5 ppm detection limit were observed upon introduction of MoO₃ on NPS. The sensors were found to be repeatable and stable for almost one year, as tested under humid condition at room temperature.

Reference is made to a non-patent literature ‘Fabrication of TiO₂ Nanostructures on Porous Silicon for Thermoelectric Application, F. N Fahrizal et al, AIP Conference Proceedings 1883, 020031 (2017); doi: 10.1063/1.5002049’ that discloses the fabrication of TiO₂ Nanostructures on Porous Silicon for Thermoelectric Application and discusses the growth of TiO₂ on porous silicon. But it does not relate to MEMS heater fabrication. However, the non-patent literature document not talk about MEMS heater fabrication and it only discusses growth of TiO₂ on porous silicon.

Reference is made to ‘Planarization of the porous surface of composition “nanoporous silicon dioxide-titanium dioxide” by atomic-molecular chemical assembly, Victor V. Luchinin et al, Atomic Layer Deposition (BALD), International Baltic Conference, 2-4 Oct. 2016; doi: 10.1109/BALD.2016.7886522’ that discloses the deposition of TiO₂ on SiO₂ using ALD. But it does not relate to MEMS heater fabrication.

Reference is made to ‘Site-specific and patterned growth of TiO₂ nanotube arrays from e-beam evaporated thin titanium film on Si wafer, Karumbaiah N Chappanda et al, 2012 IOP Publishing Ltd, Nanotechnology, Volume 23, Number 38’ that mentions growth of TiO₂ nanotubes on Ti thin film deposited on Si wafers. But this also does not relate to MEMS heater fabrication.

Metal oxide based gas sensor are known for operating at much elevated temperatures which results in higher power consumption (in order of hundreds). This drawback limits their application in battery operated devices as well as system on chip (SOC) applications.

Therefore, summarizing the drawbacks of the existing techniques as discussed above, as follows:

1. High operating temperature of metal oxide based sensors

2. High power consumption from metal oxide based sensors

3. Poor selectivity towards alcohol

Metal oxide based gas sensors are known for operating at much elevated temperatures which requires high power consumption (in order of hundreds). This drawback limits their application in battery operated devices as well as system on chip (SOC) applications.

Thus, in view of the drawbacks of the prior art, there is a dire need to develop an optimal operating temperature operated metal oxide based sensors, with low power consumption and which has reliable sensitivity towards alcohol. The fabrication of a planar MEMS heater with nano-silicon and TiO₂ heterojunction that is useful as battery operated gas-sensor is not known in the art.

### SUMMARY OF THE INVENTION

The following disclosure presents a simplified summary of the invention in order to provide a basic understanding of some aspects of the invention. This summary is not an extensive overview of the present invention. It is not intended to identify the key/critical elements of the invention or to delineate the scope of the invention. Its sole purpose is to present some concept of the invention in a simplified form as a prelude to a more detailed description of the invention presented later.

An objective of the present invention is to overcome the drawbacks of the prior art as mentioned above.

Another objective of the present invention is to provide a method of simpler fabrication (in less number of steps) of micro-heater platform for Nano-Si and metal oxide sensor.

Yet another objective of the invention is to display low power consumption.

Another objective of the invention is to develop a room temperature or near room temperature operable metal oxide based sensor integration with MEMS.

Still another objective of the invention is to fabricate a battery operated planar MEMS technology for sensing applications.

Accordingly, in one aspect, the present invention provides a method of fabrication and characterization of a wafer scalable, planar MEMS heater integration with TiO₂/Nano-Si as an ethanol sensing platform. Nano-Si TiO₂ is introduced, which contains the porous morphology with significant depth. Due to introduction of Nano-Si in micro heater fabrication the power consumption of the complete chip is significantly reduced. This significant change in power consumption is due to the thermal conductivity of Nano-Si.

Another aspect of present invention relates to a method of fabrication of the MEMS integrated sensor, the method comprising the steps:
a. obtaining a double sided polished (DSP) p type<100> Si wafer having resistivity 1-10 Ωcm;
b. growing of thermal oxides of 1 μm at 1100° C. for insulation and passivation of devices;
c. fabricating nano-Si on wafer using electrolytic solution of HF: C₂H₅OH in 1:1 to 1:4 ratios;

d. etching of complete chrome gold using standard Cr/Au etchants in order to pattern Ni heater;
e. depositing of 0.4 μm Ni/Cr thin film followed by etching selectively using same mask of heater and IDE structure;
f. depositing of TiO₂ followed by selective lifting off of TiO₂ using a photoresist; and, finally
g. performing of alignment to make a backside cavity front to back for opening of 50-270 μm from the back side.

Briefly, various aspects of the subject matter described herein are directed towards a fabrication process and particularly towards a scalable and working temperature for optimized fabrication process related to MEMS technology.

Other aspects, advantages, and salient features of the invention will become apparent to those skilled in the art from the following detailed description, which, taken in conjunction with the annexed drawings, discloses exemplary embodiments of the invention.

### BRIEF DESCRIPTION OF THE ACCOMPANYING DRAWINGS

The above and other aspects, features, and advantages of certain exemplary embodiments of the present invention will be more apparent from the following description taken in conjunction with the accompanying drawings in which:

FIGS. 1(a) to 1(i) illustrate the step wise micro-fabrication process flow for TiO₂/Nano-Si based sensor using planar MEMS technology in accordance with one implementation of the present invention.

FIG. 2 illustrates the top schematic view of the MEMS integrated low power operated sensor in accordance with one implementation of the present invention.

FIG. 3(a) illustrates Simulated temperature distribution of different micro-heaters with nano-Si at 1.5 V: (a) heater design A-D_{A} in accordance with one implementation of the present invention

FIG. 3(b) illustrates Simulated temperature distribution of different micro-heaters with nano-Si at 1.5 V: (b) heater design B-D_{B} in accordance with one implementation of the present invention.

FIG. 3(c) illustrates Simulated temperature distribution of different micro-heaters with nano-Si at 1.5 V: (c) heater design C-D_{C} in accordance with one implementation of the present invention.

FIG. 3(d) illustrates Simulated temperature distribution of different micro-heaters with nano-Si at 1.5 V: (d) D_{C} without nano-Si in accordance with one implementation of the present invention.

FIG. 3(e) illustrates Isothermal profiles for all design in accordance with one implementation of the present invention

FIG. 3(f) illustrates comparison of power in accordance with one implementation of the present invention

FIG. 4(a) illustrates the SEM micrographs of the fabricated device in accordance with one implementation of the present invention.

FIG. 4(b) illustrates EDS spectrum of TiO₂/Nano-Si in accordance with one implementation of the present invention.

FIG. 5 illustrates the IR imaging of micro-heater at 1.5 V, and temperature versus distance profile in accordance with one implementation of the present invention

FIG. 6(a) illustrates the selectivity test in presence of different analytes, in accordance with one implementation of the present invention.

FIG. 6(b) illustrates the Sensor operating temperature test, in accordance with one implementation of the present invention.

FIG. 6(c) illustrates the sensor response (%) with exposure to different analytes and their respective concentrations, in accordance with one implementation of the present invention.

FIG. 6(d) illustrates the sensor response (%) with respect to number-of days, in accordance with one implementation of the present invention.

Persons skilled in the art will appreciate that elements in the figures are illustrated for simplicity and clarity and may have not been drawn to scale. For example, the dimensions of some of the elements in the figure may be exaggerated relative to other elements to help to improve understanding of various exemplary embodiments of the present disclosure. Throughout the drawings, it should be noted that like reference numbers are used to depict the same or similar elements, features, and structures.

### DETAILED DESCRIPTION OF THE PRESENT INVENTION

The following description with reference to the accompanying drawings is provided to assist in a comprehensive understanding of exemplary embodiments of the invention. It includes various specific details to assist in that understanding but these are to be regarded as merely exemplary.

Accordingly, those of ordinary skill in the art will recognize that various changes and modifications of the embodiments described herein can be made without departing from the scope of the invention. In addition, descriptions of well-known functions and constructions are omitted for clarity and conciseness.

The terms and words used in the following description are not limited to the bibliographical meanings, but, are merely used by the inventor to enable a clear and consistent understanding of the invention. Accordingly, it should be apparent to those skilled in the art that the following description of exemplary embodiments of the present invention are provided for illustration purpose only and not for the purpose of limiting the invention as defined by their equivalents.

It is to be understood that the singular forms “a,” “an,” and “the” include plural referents unless the context clearly dictates otherwise.

By the term “substantially” wherever used or will be used later it is meant that the recited characteristic, parameter, or value need not be achieved exactly, but that deviations or variations, including for example, tolerances, measurement error, measurement accuracy limitations and other factors known to those of skill in the art, may occur in amounts that do not preclude the effect the characteristic was intended to provide.

Features that are described and/or illustrated with respect to one embodiment may be used in the same way or in a similar way in one or more other embodiments and/or in combination with or instead of the features of the other embodiments.

It should be emphasized that the term “comprises/comprising” when used in this specification is taken to specify the presence of stated features, integers, steps or components but does not preclude the presence or addition of one or more other features, integers, steps, components or groups thereof.

In one implementation, the present invention relates to a development of scalable planar MEMS technology process for low power operated selective ethanol sensor is being presented. A wafer scale process is developed for the fabrication of MEMS integrated sensors. It can be observed that in comparison to crystalline silicon platform (which is a conventional approach) the power consumption of the nano-silicon based platform was selectively. This device structure can be based on a battery operated planar MEMS technology as its power consumption is much lesser.

In the implementation, the Micro heater and IDE structures can be made of Ni with power consumption of 18 mW, but not limited to it, with a uniform heating in sensing area. For comparison, the c-Si based sample have been simulated which showed power consumption of 30 mW. After fabrication, the sensor has been tested for optimum operating temperatures and in presence of different analytes.

In the implementation, due to introduction of Nano-Si in micro heater fabrication, the power consumption of complete chip significantly reduced. This significant change in power consumption is due to thermal conductivity of Nano-Si. The sensor material may consist of a heterojunction comprising of porous silicon and titanium dioxide. This MEMS integrated sensor platform will show a change in resistance upon adsorption of gas/vapour molecules. For making a comparison to crystalline Si, both types of structures can be simulated using COMSOL MEMS module (introducing porous media).

### Fabrication of MEMS Based Sensor

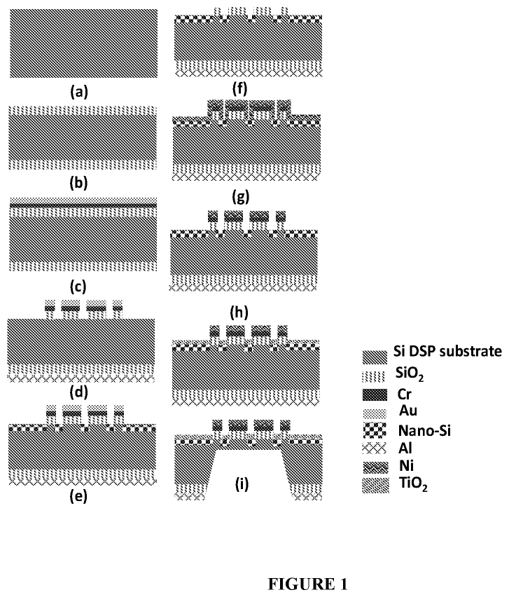

In one implementation, in the present invention, MEMS micro-heater platform with Nano-Si that provides exceptional prospects for sensors is disclosed. The step wise process for fabrication of MEMS planar platform for TiO₂/Nano-Si based sensor as per the present invention is shown in FIG. 1(a)-(i).

In the implementation, the method of fabrication of the MEMS planar platform as per the present invention begins with a 2″ double sided polished (DSP) p type<100> Si wafer having resistivity 1-10 Ωcm. Since, process is scalable so 2″, 4″ or 6″ wafers may also be used.

In one implementation, for insulation and passivation of devices 1 μm thermal oxides can be grown at 1100° C. using quartz furnace as displayed in FIG. 1(b). A thin film of Cr/Au (10/100 nm) can be deposited and patterned for defining the nano-Si fabrication region (as shown in FIG. 1(c-d)). After that Nano-Si was fabricated (anodization technique) on patterned wafer using electrolytic solution of HF:C₂H₅OH in 1:1 to 1:4 ratios. This anodization can be performed at current density 20 mA cm⁻² for duration of 30 secs to 10 minutes (FIG. 1(e)). The current density may vary from 5-100 mA·cm⁻² and time may vary from 30 secs to 120 sec. In order to pattern Ni heater, the complete chrome gold can be etched using standard Cr/Au etchants as demonstrated in FIG. 1(f)). A 0.4 μm Ni/Cr thin film can be deposited and etched selectively using same mask of heater and IDE structure as shown in FIGS. 1(g)-(h). TiO₂ (for example, 10 nm) is deposited and then a selective lift-off of TiO₂ is performed using a photoresist. In order to make a backside cavity front to back alignment was performed for opening of 100 μm cavity (this may vary from 50-270 μm) from the back side.

In the implementation, the fabrication method as mentioned above is scalable fabrication and compatible with semiconductor fabrication process. The micro-heater and sensing area (IDE) are made on same plane, therefore it is also known as the planar MEMS technology.

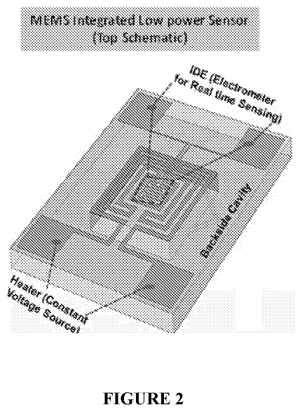

In one implementation, FIG. 2 illustrates the Nano-Si based Sensor using planar MEMS technology.

### Simulation of Micro-Heater

In one implementation, the structure of micro-heater can be designed using MEMS-CAD Tool, Comsol Multiphysics, but not limited to it. This software to can be mainly used to analyse temperature uniformity, mechanical stability, power consumption and thermal stability of device.

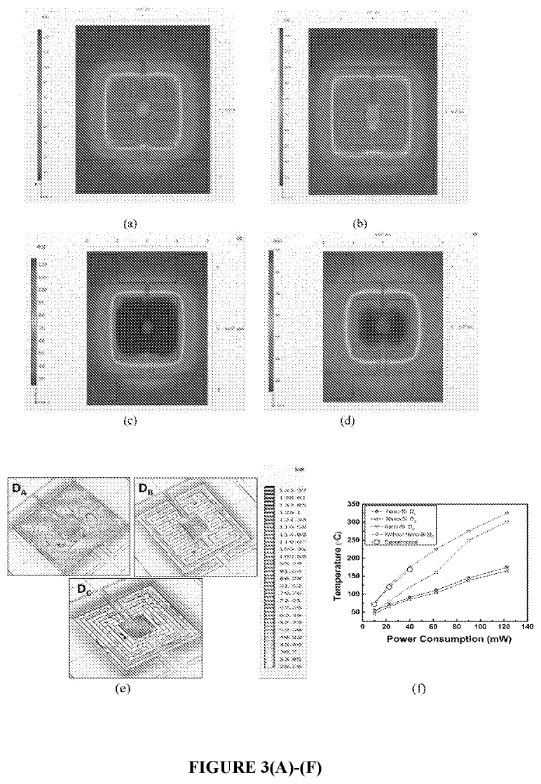

In an exemplary embodiment, the structural dimensions were taken as follows: heater strip width, 100 μm; gap between heater strips, 100 μm; heater size, 2000 μm×2000 μm; dielectric membrane thickness, 1 μm, and Ni thickness, 0.4 μm. However, the structure is not limited to this dimensions. For these simulations, to include the effect of convection, the air velocity above the micro heater can be taken as 2×10⁶ μm/s but not limited to it. The height of the air column beyond which the temperature quickly decreases to ambient temperature is taken to be approximately equal to the length of the diagonal of the micro heater area over the cavity. For the simulation of the nano-Si-based micro-heater, the physical model used was heat transfer in porous media under the ‘heat transfer in solids’ physics. In finite element analysis the micro-heaters area was kept constant, and different designs (D_{A}, D_{B}, and D_{C}) of the heaters were used for analyzing the temperature profiles (FIGS. 3(a)-(c)). These temperature profiles were used to confirm the temperature distribution in the sensing area (IDE area). All micro-heater designs were optimized on the nano-Si platform with an applied voltage range of 1-5 V. The optimized heater design was also tested on the crystalline Si platform (FIG. 3(d)). The sensor was optimized at 100° C., and thus the objective was to reach this temperature at a minimal voltage with a uniform temperature distribution in the sensing area. The design of FIG. 3(c) shows the heater temperature to be around 100° C. with uniform heating in the sensing area at 1.5 V. It is also evident from FIGS. 3(a) and (b) that the temperature distribution is not very uniform and is less than 100° C. in the sensing area at the same applied voltage. The average temperature at the sensing area was 70.2° C. and 65.2° C. for designs D_{A} and D_{B} respectively. This is due to the bending and corners in the designs where the charge gets accumulated and more heat get dissipated from these ‘hotspots’ due to Joule heating. This does not allow the temperature to rise more than a certain extent in these designs. In contrast, D_{C} has less number of such ‘hotpots’ and thus allows the temperature of the sensing area to rise to around 100° C. A similar phenomenon was noticed when the isothermal profiles were captured for all three designs in COMSOL (FIG. 3(e)). These also express that the temperature is less, and its distribution is non-uniform in D_{A} and D_{B}. The isothermal profile of DC displays the heating spreading uniformly from the center outward, and the temperature in the sensing area reaches the optimized temperature of the sensor. The non-uniformity in the temperature distribution can also be understood from the resistance—temperature relationship of a metal, as shown below

R=R₀(1+α(T−T₀)).

where, R₀ and R are the resistances at room temperature T₀ and at an elevated temperature T, respectively, and α is the temperature coefficient of resistance. This coefficient for Ni is positive, meaning that its resistivity increases with a rise in temperature due to phonon scattering, which reduces the electron mean free path. Minimum variation in R with T in D_{C} design may be the reason for achieving uniform temperature in the sensing area. Thus, design D_{C} is the optimized design and gave the best results on the nano-Si platform. FIG. 3(d) depicts the simulation result of the c-Si platform based micro-heater design D_{C} with a maximum temperature of 75° C. attained at 1.5 V. According to Joule heating, this change in applied voltage and temperature is directly related to the power consumption of the micro-heater. The resistance of the fabricated microheater was measured using a Keithley 4200A-SCS Parameter Analyzer as ˜100Ω, which was taken into account for the power consumption calculation. The trend of temperature attained with power consumption in both nano-Si and without nano-Si is given in FIG. 3(f). In the case of nano-Si, the consumption was 18 mW with a uniform heating in the diaphragm area. For comparison, the c-Si-based sample was also simulated; it showed a power consumption of 33 mW, almost double that of nano-Si. This confirms that the power consumption was much lower in the case of nano-Si. This is probably due to the lower thermal conductivity of the nano-Si (works as glass) compared to the c-Si. This further indicates that the micro-heater plays an important role in deciding the power consumption of the gas sensor. The experimental values were found to be very close to simulated values (FIG. 3(f)). The slight gap between them pertains to the small difference between the experimental values of the resistance obtained from the fabricated device and values picked up from the simulations. The simulation result also reveals that at the sensing area the temperature distribution is quite uniform with a low power consumption of 18 mW. Introducing nano-Si with MEMS planar technology played a key role in the reduction in power consumption.

The thus produced micro-heater platform attributes a nano-porous morphology of TiO₂ ranging from ˜4-6 nm and the depth of Nano-Si, which is almost ˜7 μm. The heterostructure of TiO₂/Nano-Si is responsible for the preparation of selective and stable sensor with optimum response at 100° C. The MEMS planar platform for TiO₂/Nano-Si as per the present invention shows low power consumption as compared to prior art because temperature distribution therein is quite uniform. Since, the power consumed by the disclosed device is extremely less it can be used as a battery powered device (low power operated).

### Morphological and Structural Characterization of TiO₂/Nano-Si and Heater

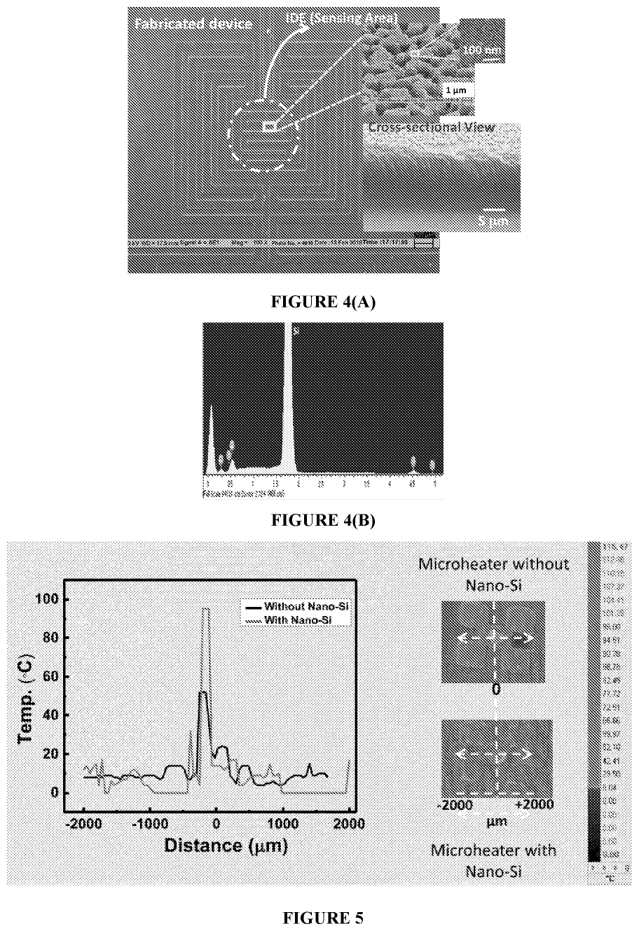

In one implementation, FIG. 4(a) shows the micrographs of the fabricated MEMS integrated TiO₂/Nano-Si based sensor platform. It is clear from the results obtained from the SEM that micro-heater and sensing area (IDE) are made on same plane. The inset of FIG. 4(a) shows the morphology and cross-sectional view of the same sample at higher magnification. It attributes a nano-porous morphology of TiO₂ ranging from ˜4-6 nm. The cross-sectional view depicts the depth of Nano-Si, which may be almost ˜7 μm. Since this morphology is uniformly observed across the wafer, the process can be used for fabrication can be used for larger silicon wafers too with the properties of films remaining the same and so the disclosed process is scalable. FIG. 4(b) depicts the EDS spectrum of TiO₂/Nano-Si sample. It clearly demonstrates the overlap of the peaks Ti and O at energy <2 keV. At higher energy levels (4˜6 keV) two isolated Ti peaks were observed which confirms the formation of TiO₂ on Nano-Si surface using reactive RF sputtering using no substrate heating.

FIG. 5 represents the temperature profile versus distance graph for both samples (with and without nano-Si). It shows that the temperature profile, obtained by the IR camera measurement, is almost constant in the active region (sensor area) across the heater filament. The heater temperature is maximum near the center and almost at ambient temperature over the outside area of the suspended filaments. There is an observable change in IR profiles at the center region of both samples, which was observed due to the difference in micro-heater platforms viz. nano-porous and crystalline Si. FIG. 5 explains that a maximum temperature of ˜100° C. was attained in the center when nano-Si was used as the platform. Conversely, only ˜55° C. was attained when c-Si was used. The pictures captured from the IR camera are shown with axes to understand the distances.

### Sensor Testing

In one implementation, sensor response %, (S %), of a sensor is defined as the ratio of change of electrical resistance in presence of test analyte, to the value of base resistance in air,

S%=(R_{g}−Rₐ)/Ra×100 (1)

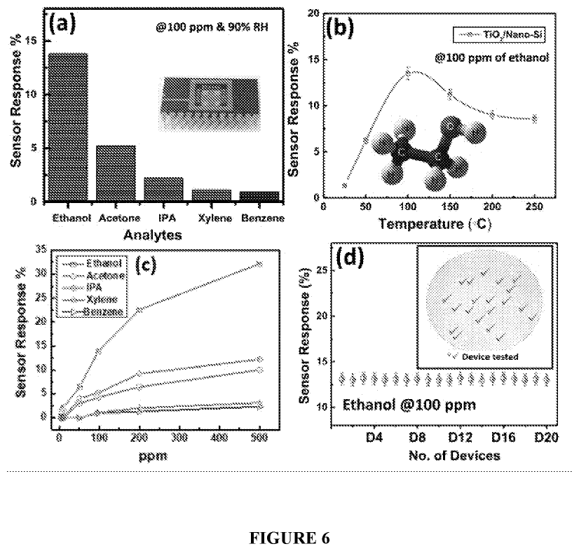

In one implementation, concentration of ethanol vapours (ppm) can be controlled using relation between ppm and vapour pressure of the analyte. The sensor response was tested for a wide range of ethanol vapours 0.5 ppm to 100 ppm at 90% RH. TiO₂/Nano-Si sample used in the present invention is found to be most responsive for ethanol in comparison to other VOCs that includes but not limited to, iso-propyl alcohol (IPA), acetone, xylene and benzene (FIG. 6(a)). This shows the role of heterostructure of TiO₂/Nano-Si towards preparation of selective and stable sensor with optimum response at 100° C. The FIG. 6(b) shows the optimum operating temperature test of TiO₂/Nano-Si sensor, specifically FIG. 6(b) confirms the operating temperature of sensor as 100° C. FIG. 6(b) illustrates the sensor response % versus ppm at 100° C. for a range ppm of different volatile organic compounds (VOCs). The graphical illustrations show that sensor response is following linear response with increase of analytes concentration. Reference is made to FIG. 6(c) wherein the response of the sensor is substantially linear till 200 ppm of ethanol and then a saturation is reached beyond 200 ppm. The fabrication process was scalable and to prove this, sensors were picked up from different locations of the wafer and the sensor response (%) was found to be almost similar (with 5% variation (FIG. 6(d)) as obtained from these sensors.

### Advantages of the Present Invention

Some of the non-limiting advantages of the present invention are mentioned below:
1. The introduction of Nano-Si in micro heater fabrication, significantly reduces the power consumption of the complete chip.
2. Due to its low power consumption, it can be used in battery operated system.
3. The MEMS integrated sensor platform as per the present invention is easy to integrate for SOC, is of relatively low cost, has high surface area, has porosity, is significantly sensitive, and possess IC compatibility.
4. Excellent integration of micro heater platforms and nanostructured materials, with low power consumption.

Although a method for fabrication of MEMS integrated sensor and a sensor thereof been described in language specific to structural features and/or methods, it is to be understood that the embodiments disclosed in the above section are not necessarily limited to the specific features or methods or devices described. Rather, the specific features are disclosed as examples of implementations of a method for fabrication of MEMS integrated sensor and a sensor thereof been.

1. A method of fabrication of MEMS integrated sensor, the method comprising the steps:
a. obtaining a double sided polished (DSP) p type<100> Si wafer having a resistivity 1-10 Ωcm;
b. growing thermal oxides of 1 μm at 1100° C. for insulation and passivation;
c. fabricating nano-Si on the wafer using an electrolytic solution of HF:C₂H₅OH in 1:1 to 1:4 ratios;
d. etching complete chrome gold using standard Cr/Au etchants pattern a Ni heater;
e. depositing 0.4 μm Ni/Cr thin film followed by etching selectively using a mask of heater and IDE structure;
f. depositing TiO₂ followed by selective lifting off of TiO₂ using a photoresist; and,
g. performing alignment to make a backside cavity front to back for opening of 50-270 μm from the back side.

2. The method of fabrication of the MEMS integrated sensor as claimed in claim 1, wherein the Si wafers are 2″, 4″, or 6″.

3. The method of fabrication of the MEMS integrated sensor as claimed in claim 1, wherein the method comprises a step of depositing a thin film of Cr/Au (10/100 nm) and patterning for defining nano-Si was fabrication region.

4. The method of fabrication of the MEMS integrated sensor as claimed in claim 1, wherein the backside cavity has opening of 100 μm.

5. The method of fabrication of the MEMS integrated sensor as claimed in claim 1, wherein the nano-Si is fabricated by anodization technique.

6. The method of fabrication of the MEMS integrated sensor as claimed in claim 4, wherein anodization is performed at current density 5-100 mA·cm⁻².

7. The method of fabrication of the MEMS integrated sensor as claimed in claim 4, wherein anodization is performed at a current density 20 mA·cm⁻².

8. The method of fabrication of the MEMS integrated sensor as claimed in claim 4, wherein anodization is performed for 30 secs to 10 mins.

9. The method of fabrication of the MEMS integrated sensor as claimed in claim 4, wherein anodization is performed for 10 minutes.
